(19) United States
(12) Patent Application Publication
(10) Pub. No.: US 2025/0302378 A1
(43) Pub. Date: Oct. 2, 2025

(54) METHOD FOR MONITORING THE SLEEP OF A USER, AND CORRESPONDING MONITORING DEVICE AND COMPUTER PROGRAM

(71) Applicant: ORANGE, ISSY-LES-MOULINEAUX (FR)

(72) Inventor: Grégoire Lefebvre, Châtillon (FR)

(21) Appl. No.: 18/864,076

(22) PCT Filed: May 9, 2023
(86) PCT No.: PCT/EP2023/062187

§ 371 Date: Nov. 8, 2024

(30) Foreign Application Priority Data
May 10, 2022 (FR) FR2204436

(51) Int. Cl. A61B 5/00; A61B 5/145

(52) U.S. Cl. CPC A61B 5/4812; A61B 5/14542; A61B 5/746

(57) ABSTRACT

A method for monitoring the sleep of a user using at least one user device located in proximity to the user and/or worn thereby, the at least one user device being connected to a communication network. This method includes: determining at least one curve representative of a time sequence of sleep stages during a current time period, referred to as a current sleep signal; obtaining an indicator of disturbance of the sleep of the user, this including a first comparison of the current sleep signal to a set of curves representative of time sequences of sleep stages determined for a reference time period, referred to as reference sleep signals; and making a decision as to whether to send an alert notification to the communication network, this decision being made depending on the indicator of disturbance of sleep and on at least one decision criterion.

### FIELD OF THE INVENTION

The invention relates mainly to home-support services for vulnerable people (for example: seniors, people suffering from deficiencies, people suffering from a chronic disease, etc.). However, the invention is also aimed at any people desiring to monitor the evolution over time of their sleep at home.

In particular, the invention is aimed at a better evaluation of the independence of people, in particular vulnerable people, by monitoring the evolution in their sleep via an actimetry system for the implementation of a remote monitoring service.

### PRIOR ART

The loss of independence, in particular among vulnerable people, is an important issue for the coming years. In the context of home-support services for these people, it is necessary to be able to monitor that they are capable of remaining alone in their home.

In this respect, it is known that sleep disorders, such as a lack of sleep, or on the contrary, too much somnolence, affect people's health and thus, eventually, their independence.

There are six main categories of sleep disorders, related to diseases or not:
insomnias;
respiratory disorders during sleep (for example: sleep apnea);
hypersomnias of central origin (for example: narcolepsy, idiopathic hypersomnia, Klein Levin syndrome, etc.), which are not related to a disorder of the circadian rhythm, respiratory rhythm or other cause of disorders of nocturnal sleep;
disorders of the circadian rhythm, that is to say a desynchronization between the internal wakefulness-sleep rhythms and the light-darkness cycle;
parasomnias (of the sleepwalking type for example);
abnormal movements related to sleep (for example: restless legs syndrome).

In the case of vulnerable people, the appearance among these people of such sleep disorders, whether they are related to a pathological state or not, can call into question their keeping at home.

Indeed, sleep disorders can reveal variations or lacks in restful sleep, and consequently indicate a loss of independence in the vulnerable person.

In another context, among non-vulnerable people, sleep disorders can lead to an alteration in psychological functions, a reduction in the efficiency of the immune system, but also increase the risks of developing more serious diseases for example such as cardiovascular diseases. Consequently, among these people, sleep disorders can also lead to a loss of independence, which will have different consequences relative to vulnerable people however (for example: loss of awareness, difficulties at work, etc.).

Generally, every evening we fall asleep and we wake up the next day also approximately at the same time, since our sleep rhythm is regulated by our brain.

Thus, during sleep, there is a succession of five different stages of sleep:
(1) the awake stage (called AWK stage), which is the awake phase;
(2) the stage called N1, which is a stage of transition between wakefulness and sleep. The sleeper does not really have the impression of sleeping, they doze;
(3) the stage called N2, which is the stage of confirmed sleep. An electroencephalogram recorded during sleep shows characteristic figures that allow to affirm that the sleeper is sleeping;
(4) deep sleep, or stage called N3. This stage is characterized, for example, by slow and ample waves on an electroencephalogram, which gives it its name of slow-wave sleep. It is a deep sleep from which it is difficult to wake the sleeper;
(5) paradoxical sleep (or stage called NR), during which brain activity is intense, rather close to that of wakefulness, there are very fast, jerky eye movements.

Typically, falling asleep is followed by light sleep (stage N1 then stage N2) that leads on average in about twenty minutes to deep slow sleep (stage N3). After approximately 90 minutes, paradoxical sleep appears (stage NR). These various stages constitute the first sleep cycle. A cycle lasts approximately 90 to 100 minutes. One night includes 4 to 6 cycles, according to the length of the sleep. The first half of the sleep is particularly rich in deep slow sleep, while the second half substantially consists of the alternation of light sleep and paradoxical sleep.

Hereinafter, “sleep” means the set of sleep cycles each comprising a specific sequence of the various stages of sleep, during the same night. In other words, sleep is structured by a certain number of cycles, each cycle consisting of different stages of sleep through which the sleeper passes.

In order to analyze and characterize the sleep of people (for example: the number of cycles per night, type of stage forming a cycle, duration of each stage, etc.), various physiological parameters representative of the sleep can be monitored during the night. The variations in measurement of these physiological parameters are indeed characteristic of the various stages of sleep through which the sleeper passes during their night of sleep.

These various physiological parameters can be analyzed via various techniques.

Among these techniques, there is for example polysomnography, which is defined as a process of monitoring and recording several physiological parameters during sleep. Polysomnography takes into account physiological parameters such as:
the electric activity of the brain. It can be measured by electroencephalogram (EEG), using electrodes placed on the scalp of a patient;
the movements of the eyeballs, measured by electrooculogram (EOG);
muscular activity, measured by electromyogram (EMG);
cardiac activity, measured by electrocardiogram (ECG);
a respiratory effort, measured by respiratory inductance plethysmography, via the use of abdominal thoracic bands;
respiratory flow rate, measured by pneumatograph (nose and mouth mask);
the partial pressure of carbon dioxide and/or of oxygen (PaCO2/PaO2), measured by transcutaneous oximetry;
oxyhemoglobin saturation (SpO2), measured by transcutaneous oximetry;
the position of the body of the patient, analyzed by mercury sensors placed on a strap;
respiratory noises, recorded via a microphone, etc.

In another example, respiratory polygraphy is defined as being a simplified polysomnography comprising a smaller number of measured physiological parameters (but at least two parameters), most often without the neurophysiological parameters (brain activity for example). It is used especially to investigate respiratory sleep disorders. Via the analysis and the characterization of the sleep of a person, in particular via an analysis of the physiological parameters described above, it is possible to identify disturbances, or disorders, of their sleep. In particular, these techniques allow to give an accurate diagnosis and to identify diseases responsible for the sleep disorders (for example respiratory diseases such as sleep apnea). However, the techniques described above do not allow to identify and characterize with precision the sleep of a person, that is to say, the number of cycles forming their sleep, the various stages forming each cycle, the time spent in each stage and/or between each stage, etc.

In this respect, the document published by H.

Matsumoto et al., “Sleep Stage Estimation Using ECG”; IEEE “International Conference on Bioinformatics and Biomedicine (BIBM)”, 2020, pp. 2983-2983, doi: 10.1109/BIBM49941.2020.9313279, describes another technique allowing to identify the various stages that form the cycles of the sleep of a patient using signals of the ECG type that are measured then used in a decision tree that produces a staircase curve representing the stages of sleep over time (each step representing a stage) with an average classification performance only of approximately 60%.

In order to detect respiratory disorders during sleep, it is also known to use a device of the contactless radar type (“bio-radiolocation”). This device allows to estimate the respiratory movements of the thoracic cage of the patient (A. B. Tataraidze et al., “Non-contact Respiratory Monitoring of Subjects with Sleep-Disordered Breathing”, IEEE “International Conference: Quality Management, Transport and Information Security, Information Technologies” (IT&QM&IS), 2018, pp. 736-738, doi: 10.1109/ITMQIS.2018.8525001). However, this device is intrusive, stigmatizing and expensive. It is in particular not possible for a person wishing to monitor the evolution of their sleep to use it at home. Moreover, this technique for analyzing sleep does not structure the sleep finely enough to allow to estimate the restful quality of the rest that it provides.

Finally, systems allowing to analyze the sleep of a person using a device not worn by the patient are known (for example three motion sensors PIR for “Passive InfraRed”). The signals collected by these sensors are then used by a vector machine or SVM (“Support Vector Machine”), which learns to classify them (for example: movement of the eyeballs, position of the body, etc.), in 3 stages of sleep (awake, light, restful) with an accuracy of 75% (Zeng and W. Chang, “Estimation of sleep status based on wearable free device for elderly care”, IEEE “Global Conference on Consumer Electronics”, 2016, pp. 1-4, doi: 10.1109/GCCE.2016.7800530). However, the structure of the sleep in three classes remains imprecise.

Thus, at present, there is no reliable technique allowing to precisely characterize the sleeping habits of a person, and to detect changes in their sleeping habits.

In particular, none of the techniques of the prior art allows to estimate, on the basis of the detection of variations in the sleep of a person, whether the quality of their sleep degrades over time to the point of causing a loss of independence.

There is therefore a need for a technique that allows to characterize with more precision the sleep of a person on the basis of the analysis of physiological parameters representative of the sleep, in order to obtain a robust estimation of the quality of their sleep, of its evolution, or even to evaluate a potential impact on the independence of this person.

### DISCLOSURE OF THE INVENTION

The invention meets this need and proposes a method for monitoring the sleep of a user using at least one item of user equipment located in proximity to the user and/or worn by them, said at least one item of user equipment being connected to a communication network.

This method comprises:
determining at least one curve representative of a time sequence of sleep stages during a current time period, called current sleep signal;
obtaining an indicator of disturbance of the sleep of the user comprising a first comparison of the current sleep signal to a set of curves representative of time sequences of sleep stages determined for a reference time period, called reference sleep signals;
a decision to emit an alert notification in the communication network, made according to the indicator of disturbance of the sleep and at least one decision criterion.

Thus, the invention is based on a truly novel and inventive approach to the monitoring of the sleep of a user (for example: vulnerable people, or a person wishing to monitor their sleep), with a view to detecting a degradation of the quality of their sleep and deciding whether it is necessary to alert the user, a person close to them or a qualified third party (for example a doctor).

More particularly, in order to be able to propose a service to the person and adapt it to the needs of the user if necessary, the method according to the invention compares the sleep of the user during a reference period, which corresponds to a period that has already passed during which the sleep of the user was supposed to be sufficiently restful, to the sleep of the user outside of this reference period, for example such as every night outside of the reference period. For this, the method according to the invention determines: for a current period, for example such as one night, a current sleep signal, and for a reference period, a set of reference sleep signals (also called sleep history of the user).

This comparison thus allows to identify modifications of the sleeping habits of the user. In other words, it is possible to identify whether the sleep signal of the user during the current period (for example one night outside of the reference period) is different than those of their sleep history during the reference period. Differences between these signals can originate for example from: the fact that the user takes more time to fall asleep (longer AWK stage), wakes up several times per night (several AWK stages during the sleep time). More generally, this comparison allows to detect modifications in the sequences of sleep stages leading, for example, to an offset of the sleep cycles with respect to the sleep history of the user, etc. An indicator of disturbance of the sleep of the person is then estimated on the basis of this comparison.

Finally, it can be decided to emit an alert notification according to the estimation of disturbance of the sleep of the user obtained and a decision criterion. This decision criterion can be for example a threshold or several thresholds of disturbance to be exceeded, the variability of the indicator over a period of observation of several nights, etc.

Thus, the solution proposed by the invention allows to monitor the evolution in the sleep cycles of an individual over time, and to evaluate whether their sleeping habits change and in particular whether they are degrading. In particular, the invention allows to detect a degradation in the quality of the sleep of the user, but also to decide, according to the importance of this degradation, whether it is necessary to alert either the user, or people close to them, as soon as the sleep disorders detected are likely to lead to a loss of independence in this individual. Indeed, the detection of a degradation in the sleeping habits of a user can be indicative of a loss of independence of this user, who is no longer able on a day-to-day basis to maintain the conditions favorable to a sufficiently restful sleep and thus places their health in danger. This estimation of sleep disturbance is thus one of the strong indicators for evaluating the good physical, social and moral health of individuals for remote monitoring services. This estimation of disturbance of the sleep can also allow to adapt telecommunication, multimedia or home automation services, etc.

Moreover, the user, because they wear on them and/or keep one or more item(s) of equipment near them, adheres de facto to the solution for monitoring their sleep.

In one example, an alert notification can be transmitted directly to the user on an item of equipment of the connected watch type, smartphone . . . or to an eHealth service (or remote monitoring service). Thus, it is possible to put in place personal recommendation services when sleep disorders are detected and they are considered to be sufficiently significant to justify the triggering of an alert.

According to a specific feature of the invention, the determining of the current sleep signal comprises obtaining at least one current set of time sequences of measurements of at least one physiological parameter representative of the sleep of the user collected during the current time period by at least one sensor of said at least one item of equipment that is connected.

Advantageously, in order to be able to identify disturbances of the sleep of the user, at least one time sequence of measurements of these sleep physiological parameters is collected by one or more sensor(s) of the item(s) of user equipment during a current period, subsequent to the reference period, and corresponding for example to one night of sleep of the user outside of the reference period.

For this purpose, an item of user equipment continuously records one or more physiological parameters representative of the sleep, also called hereinafter sleep physiological parameters. Using one or more item(s) of user equipment worn by them (for example: connected watch, smartphone, etc.) and/or located in proximity (for example on a nightstand), measurements of these physiological parameters are collected during the sleep time (for example during one night of sleep) by one or more different, or identical, sensors of the item(s) of user equipment.

It should be noted that the sleep time of the user does not necessarily correspond to one night in the literal sense of the term. Indeed, “sleep time” means any interval of time during which the user sleeps (for example from 11 PM to 6 AM or from 8 PM to 5 AM). To simplify, hereinafter, night means a sleep time of the user.

The time sequences of current measurements thus obtained thus allow to determine a curve representative of a time sequence of sleep stages during this current period, in other words, the current sleep signal. Preferably, the measurements are collected for each night outside of the reference period. The current sleep signal of the current period (that is to say one night outside of the reference period) is then compared to the reference sleep signals of the reference period.

According to another feature of the invention, the method further comprises: determining the reference sleep signals on the basis of an obtaining of at least one reference set of time sequences of measurements of said at least one physiological parameter collected during said reference time period by at least one sensor of at least one item of user equipment connected to the communication network and located in proximity to the user and/or worn by them.

Advantageously, the method according to the invention comprises determining the set of reference signals, during the reference period (for example one week). This set of reference signals, or sleep history of the user, allows to identify sleep disturbance when they are compared to the current sleep signal.

For this, one or more item(s) of user equipment continuously record one or more sleep physiological parameters. Measurements of these physiological parameters are thus collected during the reference period by one or more different, or identical, sensors of the item(s) of user equipment.

Time sequences of measurements of sleep physiological parameters are thus obtained and used to determine for each night of the reference period a curve representative of a time sequence of sleep stages (AWK, N1, N2, N3, NR), also called hereinafter reference sleep signal. This curve, or reference sleep signal, thus represents the succession during the sleep time (for example during the night) of the various stages through which the user passes during their sleep. A reference frame, or sleep history, comprising this set of reference sleep signals is thus formed.

The item of user equipment used during the reference period can be identical or different to that used during the current period. It is thus possible for the user to use various items of equipment according to their precision or ease of use.

According to a specific aspect of the invention, obtaining the indicator of disturbance of the sleep further comprises a second comparison of the current sleep signal to a sleep signal recommended for the user.

Advantageously, the indicator of disturbance of the sleep of the user for a current period simultaneously takes into account a set of sleep signals obtained for this user during a reference period, but also a sleep signal recommended, or “ideal”, for this user. This recommended sleep signal is for example defined theoretically and represents an ideal of restful sleep for the user according to criteria related to their age, their sex, their corpulence, etc.

In other words, the invention proposes refining the estimation of the measurement of disturbance of the sleep of the user over time by comparing the current signal obtained for the individual (on-the-ground reality) to an ideal theoretical sleep signal, to estimate as correctly as possible the degradation in the quality of the sleep of the person with respect to this model.

This double comparison allows not only to take into account a change in time of the sleep of the person with respect to their personal history, but also a qualitative change of this sleep with respect to a suitable theoretical model. It thus allows to obtain a more reliable estimation of the disturbances of the sleep of the user over time and thus a more robust estimation of their independence.

According to another aspect of the invention, the method further comprises, after obtaining the indicator of disturbance, an updating of the set of reference sleep signals comprising recording the current sleep signal as reference sleep signal when said at least one decision criterion is not met.

Advantageously, it is possible to store the sleep history of an individual obtained during a reference time period and update it with the sleep signal obtained during the current period. Thus, it is possible to refine over time the sleep habits of the user and better define what a restful sleep is for the user.

In particular, only a current sleep identified as “normal” is fed into the sleep history. In other words, if the indicator of disturbance does not meet one or more decision criteria, for example by being below a disturbance threshold, the sleep is thus a good sleep, or restful sleep, representative of the independence of the user. The sleep history can thus be updated with this current sleep.

According to a specific feature of the invention, the determining of the current sleep signal, respectively of the reference sleep signals, comprises implementing an artificial intelligence module configured to associate, on the basis of a characterization model of the sleep of the user, at least one segment of the time sequences of measurements of the current set, respectively at least one segment of the time sequences of measurements of the reference sets, with at least one sleep stage.

Advantageously, the artificial intelligence module implements for example a neural network allowing to learn to associate, with segments of time sequences of physiological parameter measurements collected during the current period, respectively during the reference period, a stage of sleep (AWK, N1, N2, N3, NR). In other words, the time sequences of the current, respectively reference, sets are segmented in order to be able to associate with each segment a stage of sleep. It is thus possible to obtain a representation in the form of a curve of a sleep signal (also called hereinafter sleep curve) of the user for a given night, either during the current period, or during the reference period. In other words, the artificial intelligence module allows to characterize in a precise manner the structure and/or the duration of the sleep of the user, that is to say the succession of cycles and, inside each cycle, of the stages forming their sleep. It is thus easier to identify significant modifications in the structure of the sleep of the user.

According to another feature of the invention, the method comprises a previous learning of the characterization model on the basis of a learning database associating segments of time sequences of measurements of at least one physiological parameter representative of the sleep of a panel of users collected during a period of sleep in a controlled environment with at least one stage of sleep.

Advantageously, the learning of the sleep characterization model by the artificial intelligence module is carried out in a supervised manner on the basis of a public database associating segments of time sequences of measurements of various sleep physiological parameters with various stages of sleep to reconstruct all of the sleep cycles of the user during a night of sleep. It is thus possible to obtain a sleep signal representative of the structure and duration of the sleep of the user.

According to a specific aspect of the invention, said at least one physiological parameter is chosen from a group comprising at least: cardiac activity, brain activity, the movements of the eyeballs, muscular activity, a respiratory effort, a respiratory flow rate, a partial pressure of carbon dioxide and/or of oxygen (PaCO2/PaO2), an oxyhemoglobin saturation, a position of the body, respiratory noises.

According to another specific aspect:
the first comparison comprises obtaining a first measurement of distance between the current sleep signal and the reference sleep signals;
the second comparison comprises obtaining a second measurement of distance between the current sleep signal and the recommended sleep signal;
obtaining the indicator of disturbance comprises obtaining a weighted sum of the first measurement and of the second measurement.

Advantageously, obtaining the indicator of disturbance of the sleep takes into account the differences between the current sleep of the user and their sleep history and between their current sleep and a sleep curve recommended for this user. It is thus possible to personalize the estimation of a disturbance of sleep for each individual monitored by taking into account that a restful sleep for this individual in particular does not necessarily identically follow the theoretical curve of a recommended restful sleep.

According to another specific aspect of the invention, said at least one decision criterion comprises at least one threshold of disturbance of the sleep, the decision criterion being met when the indicator of disturbance is greater than or equal to said at least one threshold of disturbance.

Advantageously, where appropriate, when a disturbance of the sleep is noted following the comparison of the reference sleep signals, the current sleep signal and the recommended sleep signal, it is decided to emit an alert notification.

In order to limit the number of notifications emitted and to trigger such an emission only in the case of a proven and sufficiently serious disturbance of the sleep, at least one threshold of disturbance of the sleep is taken into account. It is thus possible to smooth the estimation of the disturbance of the sleep over a defined period and eliminate the disturbances that are temporary (for example related to a specific life period, etc.).

In one example, a notification is emitted when the indicator of disturbance exceeds a predetermined threshold of disturbance (for example 0.5).

In another example, a first threshold for evaluating the isolated value of the indicator of disturbance for the current night and a second threshold (which can be lower than the first) for evaluating a difference in the value of the indicator of disturbance of sleep (for example: thresholding on an Euclidean distance) with respect to an average of the values of measurements of disturbance of sleep estimated in a period of time P′ of several consecutive nights can be considered.

Advantageously, it can be decided to trigger the broadcasting of a notification if both thresholds are exceeded.

Moreover, several alert levels can be considered, according to the estimated level of disturbance (that is to say for example according to the exceeding of one or more successive disturbance thresholds (having increasing values)). For example, the lowest level (“slight” exceeding of a first level of disturbance threshold) can involve emitting personalized notifications to the monitored person to help them place themselves in the conditions for better falling asleep (for example: limiting the consumption of media/communications after a particular time of the day and/or reducing the ambient luminosity, the heating, before the time to go to sleep, etc.). At the highest alert level (exceeding of the “high” last disturbance threshold), when the disturbances are serious and are likely to cause a loss of independence of the user, one or more alert notifications are addressed to third parties.

The invention also relates to a device for monitoring the sleep of a user using at least one item of user equipment located in the proximity of the user and/or worn by them, said at least one item of user equipment being connected to a communication network.

This device is configured to:
determine at least one curve representative of a time sequence of sleep stages during a current time period, called current sleep signal;
obtain an indicator of disturbance of said sleep of said user comprising a first comparison of said current sleep signal to a set of curves representative of time sequences of sleep stages determined for a reference time period, called reference sleep signals;
decide to emit an alert notification in the communication network, made according to the indicator of disturbance of the sleep and at least one decision criterion.

The invention also relates to an item of access equipment to a communication network, comprising a device for monitoring the sleep of a user as described above.

The invention also relates to an item of user equipment comprising at least one sensor, said item of user equipment being connected to an item of access equipment to a communication network. This item of user equipment comprises a device for monitoring the sleep of a user as described above.

The invention also relates to a system for monitoring a user, comprising an item of user equipment comprising at least one sensor and being connected to a communication network, an item of access equipment to a communication network and a device for monitoring the sleep of a user as described above.

The invention also relates to a computer program product comprising program code instructions for the implementation of a method as described above, when it is executed by a processor.

The invention also relates to a recording support readable by a computer on which is recorded a computer program comprising program code instructions for the execution of the steps of the method for monitoring the sleep of a user according to the invention as described above, when said program is executed by a processor.

Such a recording support can be any entity or device capable of storing the program. For example, the support can include a storage medium, such as a ROM, for example a CD-ROM or a microelectronic circuit ROM, or a magnetic recording medium, for example a mobile support (memory card) or a hard disk or an SSD.

Moreover, such a recording support can be a transmittable support such as an electric or optical signal, which can be transported via an electric or optical cable, by radio or by other means, so that the computer program that it contains is executable remotely. The program according to the invention can in particular be downloaded on a network for example the Internet network.

Alternatively, the recording support can be an integrated circuit into which the programs are incorporated, the circuit being adapted to execute or to be used in the execution of the aforementioned method.

According to an exemplary embodiment, the present technique is implemented via software and/or hardware components. In this respect, the term “module” can correspond either to a software component or to a hardware component or a set of hardware and software components, a software component itself corresponding to one or more computer programs or subprograms or more generally to any element of a program capable of implementing a function or a set of functions.

A software component corresponds to one or more computer programs, one or more subprograms of a program, or more generally to any element of a program or of a piece of software capable of implementing a function or a set of functions, according to that which is described below for the module in question. Such a software component is executed by a data processor of a physical entity (terminal, server, gateway, decoder, router, etc.) and is capable of accessing the hardware resources of this physical entity (memories, recording supports, communication buses, electronic input/output cards, user interfaces, etc.). Hereinafter, resources mean any sets of hardware and/or software elements that support a function or a service, whether they are unitary or combined.

Likewise, a hardware component corresponds to any element of a hardware set capable of implementing a function or a set of functions, according to that which is described below for the module in question. This can be a hardware component that is programmable or with an integrated processor for the execution of software, for example an integrated circuit, a chip card, a memory card, an electronic card for the execution of a firmware, etc.

Each component of the system described above of course implements its own software modules.

The various embodiments mentioned above can be combined with each other for the implementation of the present technique.

### BRIEF DESCRIPTION OF THE DRAWINGS

Other goals, features and advantages of the invention will appear more clearly upon reading the following description, given as a simple illustrative, and non-limiting, example, in relation to the drawings, among which:

FIG. 1 presents in the form of a diagram an environment of a user during their sleep according to one embodiment of the invention;

FIG. 2 shows in the form of a diagram the steps and substeps of the method for monitoring the sleep of a user according to one embodiment of the invention;

FIG. 3 illustrates in the form of a diagram a classification module implemented for characterizing the sleep of a user according to one embodiment of the invention;

FIG. 4 illustrates, in the form of curves, sleep signals obtained after characterization of the sleep of a user by the classification module of FIG. 3, according to a first exemplary embodiment of the invention;

FIG. 5 illustrates, in the form of curves, sleep signals obtained after characterization of the sleep of a user by the classification module of FIG. 3 according to a second exemplary embodiment of the invention;

FIG. 6 schematically illustrates an example of architecture of a device for monitoring the sleep of a user in their home, according to an embodiment of the invention.

### DESCRIPTION OF THE EMBODIMENTS

The general principle of the invention is based on the monitoring of the evolution over time of the sleep of a user in order to create an indicator of disturbance of their sleep and, according to this indicator, decide whether an alert notification should be emitted.

More particularly, the invention seeks to carry out a robust estimation of the independence of a user at home by monitoring the evolution of their sleep over time. In particular, the invention proposes defining an indicator of disturbance of sleep by comparing a sleep history of this user, obtained during a reference period and stored in memory, a sleep of the user for each night (or sleep time) outside of the reference period and a “recommended” or “ideal” sleep for this user.

Thus, if the indicator of disturbance of the sleep is “slight” (for example below a predetermined threshold of disturbance), then the person is deemed to be “independent”. On the contrary, if the indicator of disturbance is “high” or increases over time (for example greater than or equal to the predetermined threshold of disturbance), this person is then deemed to be losing independence.

“Recommended” or “ideal” sleep means a sleep that is considered to be restful for the user according to standards of sleep structure and/or duration of sleep, etc. defined theoretically according to categories taking into account for example the age, the sex, the corpulence, etc. of the user. Indeed, the structure of sleep (for example type of stages of sleep, number of cycles, time spent in each stage and/or between the stages, etc.) and its duration varies over time/throughout life.

It is thus possible to create an universal reference frame defining a sleep as being restful according for example to the age of the monitored user, their sex, their corpulence, etc. For example, the “ideal” structure of the sleep of a newborn is 3 stages per cycle with 50 minutes on average per cycle and the “ideal” structure of the sleep of an adult is 5 stages per cycle with 110 minutes on average per cycle. Ideally, the 3 phases of the cycle of the sleep of a newborn are: falling asleep, active sleep, and quiet sleep. Ideally, the 5 phases of the sleep of an adult are: falling asleep, light slow sleep, deep slow sleep, another phase of light slow sleep, paradoxical sleep. For example, the duration of the sleep recommended for those over 65 years of age is from 7 to 8 h while for those that are 14-17 years old it is from 8 to 10 h.

The invention is thus based first of all on the formation of a sleep history of the user over a reference period corresponding to a succession of several nights of sleep over a given period (for example 1 week), and representing the period during which the sleep is considered to be restful, or of sufficient quality, for the monitored user (“normal” sleep for this user). This reference period is thus consequently the period during which the monitored user is considered to be totally independent. This sleep history thus represents the sleeping habits of the user. The sleep of the user during this period is also called hereinafter “reference sleep”.

This sleep history of the user is then compared to a sleep called “current” or “daily” of the user, that is to say to the sleep of this same user during a current period corresponding to a night outside of the reference period, in order to detect a possible difference.

This period called “current” is separate from and subsequent to the reference period.

This “current” sleep is also compared to an “ideal” sleep, or “recommended” sleep, for this user.

The differences detected between the current sleep, the sleep history and the recommended sleep of the user are used to evaluate an indicator of disturbance in the sleep of the user and thus deduce therefrom a possible loss of independence of this user. In other words, according to this indicator of disturbance of the sleep, it is possible to decide whether the user is losing independence and thus to alert remote monitoring services to propose personal care solutions thereto.

For this, measurement data of at least one physiological parameter of the user representative of their sleep is collected during the reference period, then during the current period, for example one night, in order to characterize the sleep (structure and/or duration) of the user during this reference period and during the current period. This data of this or these physiological parameters is obtained from measurements collected by one or more sensors integrated into one or more items of terminal equipment worn by this user, or in proximity to them (for example on a nightstand) during their sleep. This or these item(s) of terminal equipment, or item(s) of user(s) equipment, are connected to a communication network which allows them to transmit, where appropriate, one or more alert notifications to third parties in the context of a remote monitoring service.

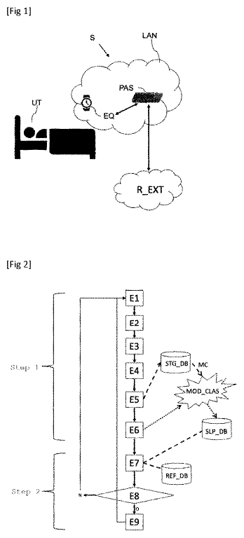

An example of an environment of a user during their sleep according to the invention will now be presented in relation to FIG. 1. This environment can correspond for example to a bedroom in the home of the user, or to a hotel room, or any place in which the user can sleep.

In order to be able to monitor the evolution in their sleep over time and independently (that is to say without assistance of the medical or technical type) the environment of the user UT comprises in particular a local communication network LAN that is managed by a residential gateway PAS connected to a data communication network R_EXT of an operator.

In this example, the network LAN is a home network, to which several items of user equipment can connect: tablets, smartphones, connected watches, connected headbands, etc.

The item of user equipment EQ is for example a smartphone or a connected object such as a connected watch, a connected bracelet, a connected headband (also called brainwaves headset) or any other item of terminal equipment equipped with an interface for communication with the network LAN and of a sufficiently small size to be worn easily by the user, or at least be in proximity to the user UT (for example placed on a nightstand).

Advantageously, the item of user equipment EQ comprises one or more sensors, identical or different, configured to measure one or more different physiological parameters of the user during their sleep. These physiological parameters can be for example one or a combination of parameters chosen from:
the electric activity of the brain, for example measured by electroencephalogram (or EEG) using a brain wave sensor comprised in an item of equipment EQ of the connected headband type worn by the user UT during their sleep;
the movements of the eyeballs, for example measured by electrooculogram (or EOG) using the camera of a smartphone of the user UT filming them during their sleep;
muscular activity, for example measured by electromyogram using a muscular sensor comprised in a connected watch or bracelet;
the cardiac activity measured by electrocardiogram (or ECG), for example via a cardiac activity sensor of a connected watch or bracelet;
respiratory noises recorded via a microphone of a smartphone of the user UT recording them during their sleep,
etc.

In other words, the sensor(s) of the item of equipment EQ are configured to continuously collect and record during the sleep of the user UT data (or signals) of one or more physiological parameters representative of the sleep of the user UT.

These sensors are also called hereinafter physiological sensors, and the data of physiological parameters is also called physiological data, or physiological signals.

In one variant, the user can carry on them, or have in proximity to them, several different items of equipment EQ (for example: a connected watch worn on the wrist and a smartphone recording them and/or filming them during their sleep) having different sensors to measure several different physiological parameters.

The item of user equipment EQ used during the reference period can be identical or different to that used during the current period.

The item(s) of user equipment EQ then transmit the physiological data collected by its or their sensors to a device DISP for monitoring the sleep of a user UT, which will be described below in relation to FIG. 6. Advantageously, this monitoring device DISP stores in a memory the measurements of the physiological parameter(s) representative of the sleep of the user thus collected.

The device DISP implements all or a part of the method for monitoring the sleep of a user according to the invention that will be described in detail below in relation to FIG. 2.

This monitoring device DISP can be embedded in the item of user equipment EQ or the plurality of items of equipment EQ.

Alternatively, the monitoring device DISP can be integrated into the gateway PAS of the user which has the advantage of benefiting from greater calculation and memory resources than the item(s) of user equipment EQ.

In another variant, the monitoring device DISP can be integrated into a server in the network of the operator R_EXT.

In the case in which the device DISP is remote from the item(s) of user equipment EQ, the transmission of the physiological data is carried out from the item(s) of user equipment EQ to the device DISP via the communication network LAN, for example via a WiFi® connection, Bluetooth®, etc.

According to the exemplary embodiment of the invention illustrated by FIG. 1, the item(s) of user equipment EQ connected to the communication network LAN, the item of access equipment to the network (gateway PAS) and the device for monitoring the sleep of a user DISP (not shown) form a system S for monitoring the sleep of a user UT.

A specific exemplary embodiment of the method for monitoring the sleep of a user according to the invention is now described hereafter.

According to this example, in order to monitor the evolution in the sleep of the user and thus their independence, two major steps are implemented:
step 1 (substeps E1 to E6) comprises:
(i) for each night during a reference period Pref, the continuous collection, the processing and the storage in a memory of a reference set comprising segments (also called series) of “reference” time sequences of measurements of one or more physiological parameters of the user representative of the sleep. In one example, the physiological parameters taken into account are parameters of brain activity (EEG) and/or of cardiac activity (ECG). Of course, other physiological parameters can be taken into account in addition to or in replacement of the. The data of this or these physiological parameter(s) is collected continuously by the sensor(s) of the item(s) of user equipment EQ as described in FIG. 1; and
(ii) the learning of a model for characterizing the sleep by a classification module configured to characterize the sleep, that is to say configured to determine the structure and/or duration of the sleep, on the basis of a labelled set of segments of time sequences of measurements, called labelled segments, of one or more physiological parameters. This physiological data is collected in a laboratory on a user panel, then processed to obtain labelled segments of time sequences that are then stored in a public database STG_DB. Advantageously, each labelled segment of time sequences of physiological measurements is associated with a stage of sleep in the database STG_DB, in order to then be able to be used to train the classification module;
(iii) the implementation of the characterization model by the classification module for the characterization of the sleep of the user for each night during the reference period Pref on the basis of the reference sets of segments of time sequences of measurements of the physiological parameter(s) of the user. It is thus possible to create a sleep history of the user, during the reference period Pref. This sleep history, representative of the sleeping habits of the user, is then stored in memory in a database SLP_DB;
step 2 (substeps E7 to E9) comprises:
(i) the collection during a current period (for example one night outside of the reference period), the processing and the storage in a memory of a set of segments of time sequences of measurements, called current set, of one or more physiological parameter(s) of the user representative of the sleep;
(ii) the implementation of the model for characterizing the sleep by the classification module for the characterization of the sleep of the user during the current period on the basis of the current set of segments of time sequences of measurements of the physiological parameter(s) of the user;
(iii) an estimation of a disturbance of the sleep of the user and thus of a possible loss of independence of the person, for the current period, for example daily, subsequent to the reference period Pref, on the basis of the sleep history of the user stored in the database SLP_DB, the “current” sleep of the current period and a “recommended” sleep, as defined above, stored in a database REF_DB.

As described above, the reference period Pref corresponds to the period during which the user is considered to be fully independent, that is to say that their sleep is considered to be of good quality and restful for this user (“normal” sleep for the user). Step 1 is thus mainly aimed at creating a sleep history of the user during this reference period, in order to be able to compare in step 2 the daily, or current, sleep of the user to this history and to a “recommended” sleep for this user.

Advantageously, this sleep history is updated with the sleep of the current period characterized by the classification module. In other words, the reference period Pref changes over time, since each new day is fed into the sleep history of the user.

More specifically, if the “current” sleep is identified as “normal” for this user, that is to say considered to be of as good quality and as restful as usual, then it is fed into the sleep history of the user SLP_DB (“current” sleep for which the indicator of disturbance of the sleep does not trigger an alert). On the contrary, if a “current” sleep is identified as “unusual”, that is to say that the indicator of disturbance of the sleep triggers an alert, then this “current” sleep is not added to the history SLP_DB to avoid denaturing its function of sleep history reflecting a “good” independence of the user.

The various substeps characterizing the main steps 1 and 2 of the method for monitoring the sleep of a user according to an exemplary embodiment of the invention will now be presented in relation to FIG. 2.

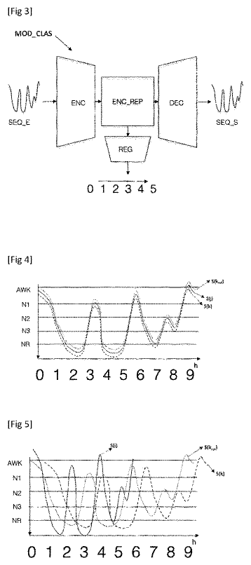

Step 1 can be decomposed into 6 substeps implemented during the reference period Pref:
a substep E1: as described in relation to FIG. 1, during the sleep of the user UT during the reference period Pref (for example: each night during a week, or a month), one or more physiological sensors of the item of terminal equipment EQ (or of a plurality of items of terminal equipment where appropriate), worn by the user, or in proximity to the latter, continuously collect (or measure) and record data, or signals, of one or more physiological parameters of the user representative of the sleep (for example: signals of the ECG and/or EEG type);
a substep E2: the physiological signals collected during each of the nights of the reference period are then each temporally cut up to obtain physiological signals of interest to be processed for each night during the reference period (from the night of day j1 to the night of day jP defining the reference period Pref, for example each night during a week where P=7 in this case) More particularly, the physiological signals are cut up to remove the signals collected at the beginning and end of a night, since the acquisition of this data by the sensor(s) of the item of equipment EQ is not indicative of the sleep at the beginning (the person puts on the item of equipment and/or places the item of equipment in proximity, gets in their bed, etc.) and at the end of the night (the person removes the items of equipment, etc.);
a substep E3: for all the physiological signals of interest associated with a day j of the reference period Pref, that is to say the physiological signals of interest obtained after the cutting up of substep E2, a window for analysis of the signals of interest is obtained. Its width is for example determined by preliminary experiments (empirical choice). In one exemplary embodiment, the physiological signals of interest cut up in substep E2 are segmented every 30 s without overlap. This segmentation is thus carried out according to sliding windows for analysis of the physiological signals of interest. A set of segments, or series, of time sequences of 30 s coming from the segmentation of the physiological signals of interest is thus obtained;
a substep E4: the physiological signals of interest obtained in E2 and segmented (for example every 30 s) in E3 to obtain a set of segments of time sequence are then processed. The processing comprises, in a non-exhaustive manner, a low-pass filtering to denoise the information, a normalization to standardize the data, a resampling of data to synchronize the sources, etc.;
a substep E5: it involves making an artificial intelligence module, for example such as the classification module MOD_CLAS presented in relation to FIG. 3, learn to classify each segment of time sequences of physiological signals of interest coming from the substeps E1 to E4 into one of the five classes corresponding to the five stages of sleep, namely the stages AWK, N1, N2, N3 and NR.

The goal being to characterize the sleep of the user during this reference period Pref, that is to say to determine the structure and/or the duration of the sleep of the user for each night during the period Pref and to thus obtain a curve of the sleep S(k) (also called sleep signal) representing the various stages of the successive sleep cycles of the user for each night k during this period Pref. In other words, substep E5 seeks to construct the sleep history of the user comprising a sleep curve S(k) of the user for each night k during this period Pref.

For this, such a learning can be supervised and be based on the database STG_DB described above. This database STG_DB is for example constructed by collecting physiological signals (for example: signals of the type EEG, ECG, EOG, etc.) from a panel of users in a controlled environment (for example a laboratory). More particularly, this database STG_DB comprises a set of segments of time sequences obtained after processing, as described in substeps E1 to E4, of the physiological signals collected.

Each segment of time sequences of physiological signals of interest of the public database STG_DB is then tagged or labelled using a piece of information identifying one of the five classes corresponding to the various stages of sleep. In one example, these tags, or labels, are of the numerical type and the segments of time sequences of data of interest (that is to say the segments of time sequences of physiological signals of interest) coming from the public database STG_DB are each associated with a label value between 0 and 4, such that:
the value 1 represents the stage N1,
the value 2 represents the stage N2,
the value 3 represents the stage N3,
the value 4 represents the stage NR, and
the value 0 represents wakefulness signals AWK.

Thus, in a learning phase, the classification module MOD_CLAS is trained in a supervised manner to classify the segments of time sequences of physiological signals of interest of the database STG_DB into one of the classes corresponding to the various stages of the sleep: AWK, N1, N2, N3 and NR.

The classification module MOD_CLAS takes as an input the segments of time sequences of physiological signals of interest of the database STG_DB and adjusts its configuration to associate with each segment of time sequences of physiological signals of interest, at the output, the stage of sleep corresponding to the label. In other words, the classification module MOD_CLAS is configured to give a value between 0 and 4 to each segment of time sequences. A sleep signal produced by the classification module MOD_CLAS with a prediction of the stage of sleep for each segment of time sequences of physiological signals of interest are thus obtained over the entire sleep, for example a sleep of 9h. Such a learning allows it to build a model for characterizing the sleep MC that it will then implement in test phase to recognize the various stages of the sleep cycles of a new set of segments of time sequences of physiological signals of the user and obtain a sleep curve S(x).

In one exemplary embodiment, the module MOD_CLAS is an artificial intelligence module that has the architecture of FIG. 3. As described above, this classification module MOD_CLAS is configured to implement the model for characterizing the sleep MC. This architecture thus takes as input data SEQ_E the segments of time sequences of physiological signals of interest of the user during their sleep (segments obtained after the substeps E1 to E4). In one example, during the learning phase, the segments of labelled time sequences of interest of the public database STG_DB are used as described above. Then, in test phase, the segments of time sequences of signals of interest are obtained from time sequences of physiological signals collected for the user and processed, as presented in relation to substeps E1 to E4, during a reference period Pref.

In substep E6, at the end of the learning phase (E5), the trained classification module MOD_CLAS takes as input the segments of time sequences of physiological signals of interest coming from substeps E1 to E4 of the reference period Pref to classify them into one of the classes corresponding to the five stages of sleep, namely the stages AWK, N1, N2, N3 and NR. Advantageously, it automatically produces as output, from the temporal succession of segments associated with a stage of sleep, a sleep curve S(k) for each night k during the reference period Pref. These sleep curves S(k) are stored in the database SLP_DB, thus forming the sleep history of the monitored user during the reference period Pref.

In one exemplary embodiment, the classification module MOD_CLAS comprises an auto-encoder ENC configured to transform the segments of time sequences of physiological signals of interest at the input SEQ_E into encoded signals to obtain an encoded compact representation ENC_REP.

Then, the classification module MOD_CLAS takes as an input the encoded representations ENC_REP and associates therewith a stage value between 0 and 5. As described above, it was previously trained to carry out such an estimation on the basis of the encoded representations ENC_REP of the input signals.

Between 0 and 1, the value represents a wakefulness state AWK. Between 1 and 2, the value represents the stage N1. Between 2 and 3, the value represents the stage N2. Between 3 and 4, the value represents the stage N3. And beyond 4, the value represents the stage NR.

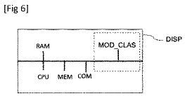

In this exemplary embodiment, finally the classification module comprises a module DEC configured to produce an output sleep signal taking the form of a curve representative of the successive stages of sleep S(x) (also noted as SEQ_S) of the user as illustrated by FIGS. 4 and 5.

The module MOD_CLAS can be for example a neural network, or any other artificial intelligence module capable of carrying out the same functions.

Step 1 ends with the formation of two knowledge bases:
the database, or sleep history, SLP_DB comprising the sleep curves S(k) of the nights k of the reference period Pref, and
the reference database REF_DB having a dimension R described above (R representing a night number k_{ref} in the database REF_DB). This reference database stores examples of sleep curves S(k_{ref}) recommended for an individual according to their age, their sex, their corpulence, etc. In other words, this database is a database representative of a sleep ideal according to the age, sex . . . category of the users.

Step 2 comprises the automatic estimation day by day (that is to say for each night j outside of the reference period Pref) of an indicator of disturbance of sleep of the person, based on the step 1 ended with the formation of the two reference frames described above (sleep history SLP_DB and reference database REF_DB) stored in memory. This last step considers a new day j, or current period, to be analyzed outside of the reference period Pref. It is decomposed into 3 substeps:
a substep E7: for at least one day j, corresponding to a current period, not belonging to the reference period Pref, and subsequent to Pref, the method according to the invention reiterates the substeps E1 to E4 of collecting and processing the physiological signals obtained by the sensor(s) (for example signals of the EEG and/or ECG type) of the item(s) of user equipment. Then, substep E6 is implemented to characterize the sleep of the user (that is to say determine their sleep curve S(j)) for the day j using the model MC used by the classification module MOD_CLAS.

FIGS. 4 and 5 show in the form of a curve various sleep signals obtained after characterization of the sleep by the classification module according to the invention.

In particular, FIGS. 4 and 5 show the various stages of sleep (AWK, N1, N2, N3 and NR) through which the user passes during their sleep time.

In FIGS. 4 and 5, the solid gray curve represents a recommended sleep signal S(k_{ref}) obtained from the database REF_DB. This sleep signal S(k_{ref}) thus corresponds to a sleep ideal for the monitored user according for example to their age, their sex, etc. The black dotted curve represents an example of a sleep signal S(k) obtained, as described above, from the sleep history SLP_DB. The solid black curve represents a current sleep signal S(j) collected for the user during the current period, obtained as described above.

The sleep signals illustrated by FIGS. 4 and 5 are characterized by a certain number of cycles, for example 4 cycles, each cycle being composed of various stages of sleep.

The reference sleep signal S(k) can be close to the recommended sleep signal S(k_{ref}) (FIG. 4), or on the contrary different (FIG. 5). Advantageously, as described below, it is possible to weight the impact of the recommended sleep signal S(k_{ref}) with respect to the reference sleep of the user S(k) in the estimation of the indicator of disturbance of the sleep of the user. Indeed, a restful sleep for the monitored user is not necessarily identical to the sleep recommended for them according to their age, etc. It is thus possible to personalize the model for estimating the sleep disturbance described below for each monitored individual.

After the determination of the sleep signals S(j), S(k) and S(k_{ref}), a calculation of the estimation of a disturbance of the sleep of the day j is carried out, noted as E(j). In one exemplary embodiment, E(j) is defined by the following equation:

$$E ( j ) = \frac{α}{P} {\sum }_{k = 1}^{P} dist ( S ( j ) , S ( k ) ) + \frac{β}{R} {{\sum }^{R}}_{{k}_{ref} = 1} dist ( S ( j ) , S ( {k}_{ref} ) )$$

with α, β weighting coefficients giving more or less importance to each component of the equation with the constraint 0<α+β<=1, and where k is one of the nights of a time period, for example such as the reference period Pref having a duration P (in number of days) and kᵣef is a night of the database REF_DB of the time period R (or set R).

The first component of the equation compares the signal, or curve, of the sleep S(j) of the day j of the current period to the sleep signals S(k) contained in the database SLP_DB (sleep history of the user).

The second component of the equation compares the signal of the sleep S(j) of the day to the sleep signals S(k_{ref}) contained in the reference database REF_DB (recommended sleep for the monitored user).

In one exemplary embodiment, a greater importance can be given to similarity with regard to the sleep history of the user with α=0.6 and β=0.4. In this example, we take β=0.4, which represents 40% of the importance of the calculation relative to the sleep signals contained in the reference database REF_DB. As a result, the differences in sleep between the day j and the sleep signals S(k) of the reference period Pref have more weight or importance that the sleep signals S(k_{ref}) coming from the reference database REF_DB (see for example FIG. 5).

Thus, if there is little difference between the sleep signals S(j) of the day j and those of the sleep history of the user S(k) during the reference period Pref, the person remains independent (see for example FIG. 4) otherwise the estimator indicates a loss of independence (see for example FIG. 5).

The distance dist between two sleep curves, or signals, S(j) and S(k) can, in one exemplary embodiment, be calculated as a DTW (from “Dynamic Time Warping”) distance. The distance is normalized between 0 and 1.

Advantageously, the estimation of sleep disturbance E(j), that is to say the indicator of disturbance of sleep, takes values between 0 and 1. If its value is close to 0, this means that the analysis of the sleep of the day is close to its history constituted in the reference period Pref and to the “recommended” references of the set R and thus the person is judged to be “independent” since the person has a behavior similar to their history and to a recommended sleep for this user (see for example FIG. 4). If on the contrary the value is close to 1, this means that the analysis of the sleep of the day differs from its history constituted in the reference period Pref and from the “recommended” references of the set R and thus the person is judged to have a “loss of independence” since the person has a behavior different to their history and to a recommended sleep for this user (see for example FIG. 5).

During a substep E8, the estimation, or indicator, of disturbance of sleep E(j) previously obtained is analyzed to decide whether the user has a loss of independence in relation to disturbances of their sleep, and whether or not it is necessary to trigger a protective action in the context of a remote monitoring service, for example such as notifying a third party of the loss of independence noted for the current period.

In a first example, the evaluation can involve always broadcasting this information. In other words, regardless of the value of estimation of sleep disturbance E(j) determined, if it is different than 0, then it is estimated to be useful to notify that there is a disturbance of the sleep and thus a potential loss of independence in substep E9 (“O” in FIG. 2), and thus the transmission of an alert notification of loss of independence in substep E9 and the proposition of a suitable service recommendation are carried out.

In a second example, it is decided to emit a notification when the indicator of disturbance of sleep E(j) meets a decision criterion, for example such as a sleep disturbance threshold. In this example, the notification is emitted when the indicator of disturbance of sleep E(j) is greater than or equal to a predefined disturbance threshold, for example equal to 0.5.

The method is reiterated for several successive current time periods and subsequent to the reference period. In one example, each current period lasts one night. The method is thus repeated each night outside of the reference period.

In a third exemplary embodiment of the invention, the decision to broadcast an alert (that is to say a notification) is taken according to the previous threshold of disturbance and at least one other decision, or relevance, criterion. For example, this other decision criterion imposes that the predefined threshold (for example 0.5) be exceeded several times over a period P′ of several consecutive nights. The indicators obtained over several consecutive nights are thus taken into account. Alternatively or in addition, this other decision criterion can take into account the differences between the values of estimation, or indicators, of disturbance obtained over the period P′ and an average of these values during the period P′. If the value of estimation of sleep disturbance E(j) varies greatly (for example: thresholding on an Euclidean distance) with respect to the average of the values of measurements of sleep disturbance estimated in the period of time P′, then the broadcasting of this notification can be decided, even if the disturbance threshold is only effectively exceeded few times over the period P′ (in this embodiment case, the values of sleep disturbance E(k′) must be calculated for all the days k′ belonging to the period P′).

If the criterion or criteria for deciding on a notification are not met (no or “N” in FIG. 2), substep E9 is not implemented and substeps E1 to E8 are directly repeated for a new current time period.

Substep E9 thus involves broadcasting a notification of information on the independence of the user according to the value E(j) estimating the sleep disturbance of the monitored person for the day j.

In one exemplary embodiment, this notification can be sent to the user to inform them of their independence, alert to a lack of independence and/or give a recommendation to gain back independence.

Alternatively, this notification can be sent to an eHealth service, to a trusted third party (family of the monitored person) or to a primary care physician. Thus, it is possible to feed applications and services related to eHealth solutions for the monitoring of vulnerable people and/or for the monitoring of inhabitants in their use of the smart home (for example such as: connected home, secured home).

In this respect, several alert levels are possible, according to the estimated level of disturbance (that is to say for example according to the exceeding of one or more successive thresholds, having increasing values, of disturbance). The lowest level (for example exceeding a first disturbance threshold) can involve emitting personalized notifications to the monitored person to help them place themselves in the conditions for better falling asleep (for example: limiting the consumption of media/communications after a certain time of the day and/or reducing the ambient luminosity, the heating, before the bedtime, etc.). At the highest level (for example exceeding of a second disturbance threshold, greater than the first threshold), when the disturbances are severe and risk causing a loss of independence in the user, one or more alert notifications are addressed to third parties.

Thus, the invention allows a robust estimation of the independence of a person at home according to their daily sleep. The invention is original in its design of a model from start to finish, that is to say from the raw data to the promotion of a suitable service, with a recognition of the sleeping habits of the user as a reference to notify eHealth services about the independence of the monitored person.

The uses are, first of all, the estimation of independence for vulnerable people in eHealth services. This estimation allows to inform the family and the medical profession of the changes in the physical, moral, and social health of the monitored person for services in particular of remote monitoring. It is possible to imagine future services that would favorably use the analysis of sleep cycles. Likewise, services adapted to the situation of an isolated worker can be provided according to their state of fatigue and of health (ex.: alert when they arrive in an area of risk for greater vigilance, etc.).

In order to illustrate more precisely the principle of the invention, FIG. 6 schematically shows the architecture of a monitoring device DISP, according to one embodiment of the invention.

In this example, the monitoring device DISP comprises a random-access memory RAM, a processing unit CPU equipped for example with a processor, and controlled by a computer program stored in a read-only memory (for example a ROM memory or a hard disk). Upon initialization, the code instructions of the computer program are for example loaded into the random-access memory RAM before being executed by the processor of the processing unit CPU.

The monitoring device DISP further comprises a memory MEM allowing in particular to store the measurements of physiological parameters of the user during their sleep using one or more sensors of the item of user equipment (or of a plurality of items of user equipment). Moreover, the memory MEM can store the databases STG_DB, SLP_DB and REF_DB.

Alternatively, these databases can be stored in a server of the operator in the outside network R_EXT (FIG. 1), or in a memory of the residential gateway PAS (FIG. 1).

The monitoring device DISP also comprises a communication module COM for the reception/transmission of measurements coming from sensors and the transmission of the alert notifications on the independence of the user.

FIG. 6 only illustrates a particular manner, among several possible, of carrying out the monitoring device DISP, in order for it to carry out at least a part of the steps of the method for monitoring the sleep of a user described in detail above, in relation to FIG. 2 in its various embodiments. In one exemplary embodiment, the monitoring device is configured to implement all of the steps of the method for monitoring the sleep of a user. For this, the monitoring device DISP comprises a classification module MOD_CLAS configured to determine the structure and/or the duration of the sleep of the user on the basis of a set of segments of time sequences of physiological measurements of interest, according to the characterization model MC learned during the learning phase described in relation to FIG. 2 (substep E5). For example, the module MOD_CLAS has the architecture described in relation to FIG. 3.

Alternatively, in another exemplary embodiment, the monitoring device DISP is configured to implement only substeps E1 to E4, then E9 to E10. The substeps E5 to E6 of learning and of characterization of the sleep of the user during the reference period Pref are thus implemented by a remote item of equipment to which the monitoring device DISP is connected, for example such as the gateway PAS that comprises the classification module MOD_CLAS described in relation to FIG. 3 and implementing the characterization model MC, or in an item of server equipment of the operator that thus comprises the classification module MOD_CLAS.

These steps can be carried out indifferently on a reprogrammable calculation machine (a PC computer, a DSP processor or a microcontroller) executing a program comprising a sequence of instructions, or on a dedicated calculation machine (for example a set of logic gates like an FPGA or an ASIC, or any other hardware module).

In the case in which the monitoring device DISP is carried out with a reprogrammable calculation machine, the corresponding program (that is to say the sequence of instructions) can be stored in a storage medium that is removable (for example such as an SD card, a USB key, a CD-ROM or a DVD-ROM) or not, this storage medium being readable partly or totally by a computer or a processor.

1. A method for monitoring sleep of a user using at least one item of user equipment located in proximity to said user and/or worn by them, said at least one item of user equipment being connected to a communication network, wherein said method is implemented by a monitoring device and comprises:
determining at least one curve representative of a time sequence of stages of sleep during a current time period, called current sleep signal;
obtaining an indicator of disturbance of said sleep of said user comprising:
(i) a first comparison of said current sleep signal to a set of curves representative of time sequences of stages of sleep determined for a reference time period, called reference sleep signals;
(ii) a second comparison of said current sleep signal to a recommended sleep signal for said user;
a decision to emit an alert notification in the communication network, made according to said indicator of disturbance of said sleep and at least one decision criterion.

2. The method for monitoring the sleep of a user according to claim 1, wherein said determining said current sleep signal comprises obtaining at least one current set of time sequences of measurements of at least one physiological parameter representative of the sleep of the user collected during said current time period by at least one sensor of said at least one item of equipment that is connected.

3. The method for monitoring the sleep of a user according to claim 2, wherein the method further comprises:
determining said reference sleep signals on the basis of an obtaining of at least one reference set of time sequences of measurements of said at least one physiological parameter collected during said reference time period by at least one sensor of at least one item of user equipment connected to said communication network and located in proximity to said user and/or worn by the user.

4. The method for monitoring the sleep of a user according to claim 1, wherein the method further comprises, after obtaining said indicator of disturbance, an updating of said set of reference sleep signals comprising recording said current sleep signal as reference sleep signal when said at least one decision criterion is not met.

5. The method for monitoring the sleep of a user according to claim 3, wherein the determining of said current sleep signal, respectively of said reference sleep signals, comprises implementing an artificial intelligence module configured to associate, on the basis of a characterization model of said sleep of said user, at least one segment of said time sequences of measurements of said current set, respectively at least one segment of said time sequences of measurements of said reference sets, with at least one stage of sleep.

6. The method for monitoring the sleep of a user according to claim 5, wherein the method comprises a previous learning of said characterization model on the basis of a learning database associating segments of time sequences of measurements of at least one physiological parameter representative of the sleep of a panel of users collected during a period of sleep in a controlled environment with at least one stage of sleep.

7. The method for monitoring the sleep of a user according to claim 2, wherein said at least one physiological parameter is chosen from a group consisting of: cardiac activity, brain activity, the movements of the eyeballs, muscular activity, a respiratory effort, a respiratory flow rate, a partial pressure of carbon dioxide and/or of oxygen, an oxyhemoglobin saturation, a position of the body, respiratory noises.

8. The method for monitoring the sleep of a user according to any claim 1, wherein:
said first comparison comprises obtaining a first measurement of distance between said current sleep signal and said reference sleep signals;
said second comparison comprises obtaining a second measurement of distance between said current sleep signal and said recommended sleep signal;
said obtaining of said indicator of disturbance comprises obtaining a weighted sum of said first measurement and of said second measurement.

9. The method for monitoring the sleep of a user according to claim 1, wherein said at least one decision criterion comprises at least one threshold of disturbance of the sleep, said decision criterion being met when said indicator of disturbance is greater than or equal to said at least one threshold of disturbance.

10. A device for monitoring sleep of a user using at least one item of user equipment located in proximity to said user and/or worn by the user, said at least one item of user equipment being connectable to a communication network, wherein said device comprises:
at least one processor; and
at least one non-transitory computer readable medium comprising instructions stored thereon, which when executed by the at least one processor configure the device to:
determine at least one curve representative of a time sequence of stages of sleep during a current time period, called current sleep signal;
obtain an indicator of disturbance of said sleep of said user comprising:
a first comparison of said current sleep signal to a set of curves representative of time sequences of stages of sleep determined for a reference time period, called reference sleep signals; and
a second comparison of said current sleep signal to a recommended sleep signal for said user;
decide to emit an alert notification in the communication network, made according to said indicator of disturbance of said sleep and at least one decision criterion.

11. Item of access equipment to a communication network, which comprises the device for monitoring the sleep of a user according to claim 10.

12. Item of user equipment comprising at least one sensor, said item of user equipment being connectable to an item of access equipment to a communication network, wherein the item of user equipment comprises the device for monitoring the sleep of a user according to claim 10.

13. A system for monitoring a user, which comprises:
an item of user equipment comprising at least one sensor and being connectable to a communication network,
an item of access equipment to a communication network, and
the device for monitoring the sleep of a user according to claim 10.

14. (canceled)

15. A non-transitory recording support readable by a computer on which is recorded a computer program comprising program code instructions for executing the method for monitoring sleep of a user according to claim 1, when said program is executed by a processor.
